(19) United States
(12) Patent Application Publication
(10) Pub. No.: US 2008/0287526 A1
(43) Pub. Date: Nov. 20, 2008

(54) Pharmaceuticals for treating or preventing oral diseases

(75) Inventor: Yao Shi, Zhongshan (CN)

Correspondence Address: BAKER & MCKENZIE LLP, Pennzoil Place, South Tower, 711 Louisiana, Suite 3400, HOUSTON, TX, 77002-2716, US

(73) Assignee: HAWLEY & HAZEL (BVI) COMPANY LIMITED, Tortola (VG)

(21) Appl. No.: 12/122,656

(22) Filed: May 16, 2008

(30) Foreign Application Priority Data
May 16, 2007 (CN) CN200710106996.4
May 24, 2007 (CN) CN200710107225.7

(51) Int. Cl. A61K 31/352; C07D 493/04; A01N 43/16; A61P 31/04; A01P 1/00

(52) U.S. Cl. 514/453; 549/382

(57) ABSTRACT

The present invention discloses a new compound of formula (I), a method of extracting the new compound, as well as various uses of the new compound in manufacturing antibacterial medicaments or oral care products or for other antibacterial uses.

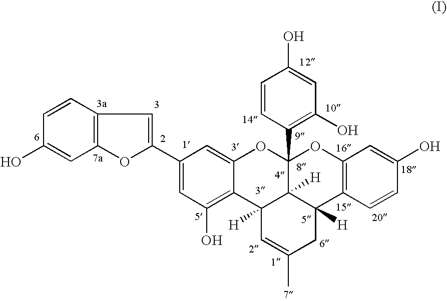

### PRIOR RELATED APPLICATIONS

This application claims priority to CN200710106996.4, filed May 16, 2007 and is incorporated by reference in its entirety.

### FEDERALLY SPONSORED RESEARCH STATEMENT

Not applicable.

### REFERENCE TO MICROFICHE APPENDIX

Not applicable.

### FIELD OF THE INVENTION

The present invention relates to a new compound Morusin A of formula (I); the pharmaceutical, oral or antibacterial products that are manufactured using Morusin A as an active ingredient; the method of preparing Morusin A; and its use in manufacturing pharmaceutical, oral or antibacterial products for use in treating or preventing oral disease or as an antibacterial.

### BACKGROUND OF THE INVENTION

Common human oral diseases include caries and periodontal disease. Caries is a chronic bacterial disease occurring in hard dental tissues, manifesting as color, shape, and qualitative changes in the dental material. Clinical symptoms of periodontal disease are gum bleeding, purulence, tooth mobility, absorption of alveolar bone and formation of periodontal pockets. Caries and periodontal disease are also one of the primary causes for the tooth loss of adults. The pathogeny of both caries and periodontal disease is now considered to be endogenous infections resulted from an imbalance in the normal oral flora caused initially by plaque and resulting in the build up of specific pathogenic microorganisms.

Bacteria are necessary for the occurrence of caries. Streptococcus mutans, Actinomyces viscosus and the like break up carbohydrates to yield acids, causing tooth inorganic material to demineralize and form caries cavities. Streptococcus mutans and Streptococcus sanguinis are also the pioneer bacteria in the formation of dental plaques, forming dental plaques together with Actinomyces viscosus and Porphyromonas gingivalis. The toxins and other detrimental substances produced by the bacteria in the dental plaques may cause the inflammatory reactions of the host, which lead to the increase in vascular permeability and the spread of inflammation; destroy the gum, dental bone and alveolar bone; and cause gingivitis and periodontal diseases.

Therefore, inhibiting oral pathogenic microorganisms is an important approach to prevent caries, gingivitis and periodontal diseases. However, products for treating caries and periodontal disease are very limited presently. On one hand, pharmaceuticals can be used to eliminate or inhibit the plaque bacteria, but the long-term use of broad-spectrum antibacterial agents will result in the selection for drug-resistant strains and other side effects. For example, the chemical plaque-controlling agent Chlorhexidine used both at home and abroad in recent years is a diguanido-hexane having obvious antibacterial function. But the long-term use of gargle containing 0.12-0.20% Chlorhexidine will cause stained tooth and formation of a brown coat at the back of tongue. On the other hand, Traditional Chinese Medicines can be used instead of pharmaceuticals, such as gargling “heat-clearing” and “detoxifying” extracts like honeysuckle, etc., as well as external-application methods, such as applying Bingpeng San (borneol and borax powder) or Asarum on the diseased part. These traditional remedies are, however, inconvenient; and there is no conclusive evidence of efficacy. Nevertheless, if the gingivitis is not treated timely or properly, the inflammation may spread to the periodontal apex, causing the inflammation of periodontal apex tissues, and affect the alveolar bone or the neighboring tissues.

What is needed in the art are new medicines from natural plants for treating common oral diseases such as caries and periodontal diseases. Oral pharmaceuticals and oral care products are required to inhibit primary oral pathogenic microorganisms, dental plaques, gingivitis, and bad breath.

Research on natural plants that can inhibit oral microorganisms has occurred throughout the world. Research has identified plants that inhibit or prevent caries, including Anogeissus Wall, Vernonia esculenta, and Garcinia morella Desv. (gamboges) of Africa and the Middle East; blood grass roots and cocoa fruit of Latin American countries; Areca catectu L. (betel nut) of Southeast Asia; and the Eucalyptus leaves of Australia. There has been no report in the prior art, however, on oral products containing Morusin A as an active ingredient.

### DESCRIPTION OF INVENTION

The present invention provides a new, pharmaceutically valuable compound Morusin A of formula (I) and a method to prepare Morusin A. Further, the present invention provides pharmaceutical or oral care product for treating or preventing oral diseases that is prepared with purified Morusin A as an active ingredient, and the use of Morusin A in manufacturing medicaments or oral care products for treating or preventing oral diseases.

By “purified Morusin A” what is meant is the pharmaceutical, oral or antibacterial product is made using a Morusin A that is at least 75% pure, preferably at least 90% pure, more preferably at least 95% pure, and even more preferably at least 98% pure, and most preferably at least 99.9% pure. Thus, the definition excludes prior art teas, or other traditional herbal preparations of the originating plant and has sufficient purity so as to be pharmaceutically acceptable. Even though a product may be made with at least 75% pure Morusin A, or preferably at least 90%, 95%, 98% or 99.9% pure Morusin A, for some pharmaceutical or oral applications, the final concentration in the ultimate product may of course be quite low, as in mouthwashes or antiseptic sprays. In other embodiments, such as tablets, the final content may be quite high.

More particularly, the invention relates to an oral product, containing purified Morusin A together with pharmaceutically or orally acceptable carriers or excipient, and optionally including additional medical adjuvant.

The oral product includes, but not limited to, oral pharmaceuticals and oral care products such as mouth wash, rinses, toothpaste, oral care gel strips, dental gums, tablets, lozenges, and the like.

Further, although the inventors have an interest in oral uses for purified Morusin A, its activity against bacteria can be employed in a variety of bactericidal or bacteriostatic uses, such as in cleansers, disinfectants, soaps, and the like, or as an antibacterial additive for tissue paper, plastics, coatings, and the like.

The present invention also provides a method for preparing the purified new compound Morusin A of formula (I). Cortex Mori root and/or bark are harvested, extracted with ethanol, then with ethyl acetate, and then subjected to silica gel chromatography and eluted with petroleum ether: acetone in 8:2 (v/v). The purified Morusin A is then formulated to make pharmaceutical, oral or antibacterial products.

The inventor of the present invention finds for the first time a new compound Morusin A of formula (I) and that it has certain inhibiting effect on oral pathogenic microorganisms. The purified compound may be applied in anti-caries and breath odor-inhibiting oral pharmaceuticals or oral care products as an active ingredient. The purified compound may also be used in oral pharmaceuticals or oral care products for countering gingivitis and dental plaques, removing oral sensitivity, countering tartar and whitening the teeth.

The invention will be illustrated in the following non-limiting examples.

### EXAMPLE 1

### Morusin A

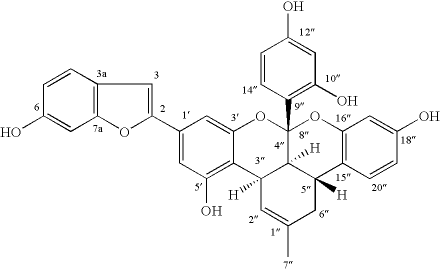

### EXAMPLE 2

### Preparation of Morusin A

50 kg Cortex Mori (White Mulberry root-bark) was extracted with 500 kg 90% ethanol under reflux for three times, three hours each time, and the extract was recovered to dryness to obtain 10.5 kg extractum. The extractum was dissolved and suspended with 8 kg hot water and 4 kg 90% ethanol. It was extracted with 50 kg ethyl acetate for three times, and recovered to dryness to obtain 4 kg ethyl acetate extractum. The resulting extractum was dissolved with acetone, adsorbed on 6 kg silica gel, and evaporated to dryness at room temperature. It was then subjected to a silica gel column chromatography (30 meshes, 30 kg) and eluted with petroleum ether: acetone (v/v, 8:2). Every 50 liters was taken as a fraction, obtaining 27 fractions (Fr.1˜Fr.27) in all. The fraction Fr.6 was subjected to a silica gel column chromatography, and eluted with chloroform:methanol (90:10) to obtain the compound Morusin A of formula (I).

The structure formula of the compound of formula (I) was determined by mass spectrometry (MS) and nuclear magnetic resonance spectrometry (¹H NMR and ¹³C NMR). An Auto SPEC 3000™ mass spectrometer was used for mass spectrometry analysis. A Bruker DRX-500™ superconductive NMR spectrometer was used to determine the nuclear magnetic resonance spectrometry of the compound, with TMS as the internal standard. The silica gel for column chromatography and thin layer column chromatography was a product of Qingdao Meigao Group Co. Ltd. Morusin A and has the following structure and is identified as formula (I):

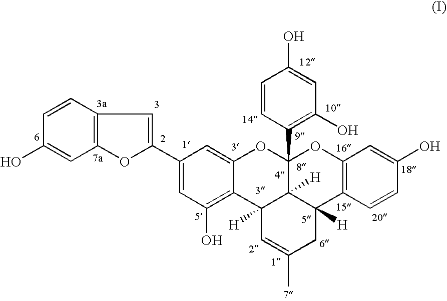

Properties: brown powder (methanol), melting point: 103-105° C.

### Molecular Formula: C₃₄H₂₆O₉

### Molecular Weight: 578

FAB-MS (−) m/z (%):561 (M+, 100). ¹H-NMR (CD₃OD, 400 MHz) δ: 7.95 (1H, d, J=8.8 Hz, H-4), 7.45 (1H, s, H-14″), 7.26 (1H, d, J=8.4 Hz, H-13″), 7.22 (1H, d, J=2.4 Hz, H-7), 7.21 (1H, s, H-20″), 7.05 (2H, d, J=2.0 Hz, H-2′, 6′), 7.02 (1H, s, H-3), 6.97 (1H, d, J=8.0 Hz, H-19″), 6.70 (1H, dd, J=9.8, 2.0 Hz, H-5), 6.13 (1H, d, J=4.0 Hz, H-2″), 4.15 (1H, dd, J=12.4, 4.8 Hz, H-3″), 3.92 (1H, s, H-4″), 3.29 (1H, m, H-5″), 2.63 (1H, dd, J=17.2, 4.8 Hz, H-6″), 2.12 (1H, dd, J=16.0, 12.0 Hz, H-6″), 1.74 (3H, s, H-7″).

¹³C-NMR (CD₃OD, 100 MHz) δ:156.7 (s, C-2), 102.0 (d, C-3), 121.0 (d, C-4), 122.5 (s, C-3a), 104.7 (d, C-5), 155.0 (s, C-6), 99.4 (d, C-7), 151.0 (s, C-7a), 133.3 (s, C-1′), 105.2 (d, C-2′), 156.7 (s, C-3′), 117.6 (s, C-4′), 161.0 (s, C-5′), 104.3 (d, C-6′), 134.8 (s, C-1″), 124.5 (d, C-2″), 36.5 (d, C-3″), 27.8 (d, C-4″), 35.8 (d, C-5″), 35.9 (t, C-6″), 23.8 (q, C-7″), 102.7 (s, C-8″), 114.2 (s, C-9″), 161.0 (s, C-10″), 161.0 (d, C-11″), 104.3 (s, C-12″), 107.2 (d, C-13″), 130.8 (d, C-14″), 117.1 (s, C-15″), 154.0 (s, C-16″), 104.6 (d, C-17″), 158.9 (s, C-18″), 110.4 (d, C-19″), 128.0 (d, C-20″).

Morusin A prepared by this method may have a very high purity. In certain examples, the purity of the Morusin A prepared by this method is higher than 90%, 95%, 98%, 99% or 99.9% as measured by common techniques, such as HPLC. In certain other examples, the concentration of the Morusin A prepared by this method is at least 50% and preferably at least 75%, 80%, or 85%.

In the examples above, we have used Cortex Mori (White Mulberry root-bark) as the plant source of the Morusin A compound. However, it is understood by those of ordinary skill in the art that other plants may also be applicable, such as those of the genus Morus, such as M. sinensis, M. alba, M. latifolia, M. serrata, M. tiliaefolia, M. bombycis, M. rotundiloba and M. laevigata, provided they contain adequate levels of the desired ingredient.

In addition to natural sources, those skilled in the art may synthesize the active ingredient easily by chemical methods without undue experimentation, to substitute the extract or purified substance used in the present invention. Therefore, those skilled in the art should understand that, in addition to extraction or purification from plants, the Morusin A of the present invention may also be achieved through chemical synthesis. Other obvious modifications of the compound disclosed in the present invention will also be included within the protection scope of the present invention.

### EXAMPLE 3

### Antibacterial Effect

The various bacterial species cultivated for antibacterial tests are listed in Table 1.

TABLE 1
Related Oral Pathogenic microorganisms

| | | Gram | Culture |
|---|---|---|---|
| Strains | ATCC number | properties | medium |
| Streptococcus mutans | 25175 | G(+) | TSB |
| (S. m) | | | |
| Porphyromonas gingivalis | 33277 | G(−) | FTM/RCM |
| (P. g) | | | (3:1) |
| Actinomyces viscosus | 27044 | G(+) | TSB |
| (A.v) | | | |

TSB = tryptone soya broth,
FTM = fluid thioglycollate medium,
RCM = reinforced clostridial medium

A single colony was picked from the Trypticase Digested Soybean peptone agar blood plate (TSA5B) of ordinarily stored bacteria species and used to inoculate corresponding broth culture medium, which was cultivated in a microaerobic environment (P.g needs anaerobic cultivation) with 95% air, 5% CO₂ at 37.0° C.±1.0° C., wherein S.m was cultivated for 18-24 h, and the remaining two species were cultivated for 40-48 h. Then corresponding broth cultivate medium was used to adjust the turbidity of the bacteria suspension to 0.5^{#} McFarland standard, equivalent to 1.0×10⁸ CFU/mL.

The bacteria were diluted through a series of 2-fold dilutions, and the anti-bacterial pharmaceutical was quantitatively inoculated with tested bacteria, and observed after incubation at 37° C. for 18-24 h, wherein the lowest pharmaceutical concentration that inhibited bacterial growth visible to the naked eye is the minimum inhibitive concentration (MIC) of the tested pharmaceuticals effective against the tested bacteria. The operating steps were:

a. Preparation of the stock solution of antibacterial pharmaceutical: 1% stock solution of various antibacterial pharmaceuticals was prepared with 100% pure ethanol as solvent. The prepared stock solution was filtered to eliminate bacteria, and was distributed into small portions to be used.

b. Range of measured concentration: the present experiment used 250 ppm as the ceiling measured concentration of the anti-bacterial pharmaceutical.

c. Measuring method: micro-dilution method.

First, 100 μL broth medium was added to each well of the 96-well plate, then 100 μL sterile anti-bacteria pharmaceutical solution, which was diluted 10-fold (1000 mg/L), was added to each well of the first column. Then each well of the first column was mixed repeatedly for 7-8 times using a multi-channel micropipet, and 100 μL sample was transferred to the second column after sufficiently mixing the pharmaceutical with TSB. After being mixed similarly for 7-8 times, 100 μL sample was transferred to the third column, and likewise to the wells of the remaining columns. Thus the concentration of the pharmaceutical was gradiently diluted 2-fold between the columns, from 500 mg/L in the first column to 0.24 mg/L in the last column (the 12th column).

The bacteria to be tested and the standard bacteria were prepared using the same method as above. Both bacteria solutions were diluted with broth medium to a bacteria content of about 10⁶ CFU/mL. Then 100 μL was inoculated into each well of the 96-well plate containing the anti-bacterial pharmaceutical. Thus the final diluted concentrations of the anti-bacteria pharmaceutical in each column were 250, 125, 62.5 . . . 0.12 mg/L, and the final inoculation quantity was about 5×10⁷ CFU/mL or 5×10⁶ bacteria in each well. The 96-well plate was placed on a microshaker and shaken for 1 minute to mix the solutions in each well. The plate was covered and sealed with adhesive paper to reduce the evaporation during the incubation process. The plate was then placed in a humid box, and cultivated in microaerobic environment with 95% air, 5% CO₂ (or in anaerobic environment with 90% N₂, 5% H₂, 5% CO₂) at 37.0° C.±1.0° C. for 18-24 h. The 96-well plate was placed under an enzyme-labeling instrument. The growth characteristics of the tested bacteria were compared with the standard bacteria to determine the MIC, which was the lowest anti-bacterial pharmaceutical concentration contained in the well without bacterial growth. The results are shown in Table 2.

TABLE 2
Effect of Morusin A on oral pathogenic microorganisms

| | A. v | S. m | P. g |
|---|---|---|---|
| | ATCC | ATCC | ATCC |
| | 27044 | 25175 | 33277 |
| Negative control (DMSO) | — | — | — |
| Positive control (Triclosan) | 3.9 | 3.9 | 7.8 |
| Compound Morusin A of formula (I) | 3.9 | 3.9 | 15.6 |

### EXAMPLE 4

### Anti-Inflammatory Effect

Anti-inflammatory activity was assessed using the KB cell (the epidermis cancer cell in human oral cavity). During the experiment, the KB cell inoculated in the cultivating plate was treated with or without positive control or compound Morusin A. After the treatment, the supernatant of the cultivating medium was collected and stored in −80° C. refrigerator. Inflammation was assessed by measuring several inflammation markers: Prostaglandin E2 (PGE₂) in the supernatant was detected by Enzyme-linked immunosorbent assay, and LUMINEX™ multifunctional liquid-phase chip analysis system was used for the detection of granulocyte monocyte colony stimulating factor (GM-CSF), tumor necrosis factor-alpha (TNF-α), and interleukins 1-beta and 6 (IL-1β and IL-6).

Prostaglandin E2 (PGE₂) result showed that the 50% inhibitory concentration of Morusin A to the growth of oral KB cell was 0.02 ppm, equivalent to that of the positive control Triclosan. The result indicated that Morusin A had significant anti-inflammatory activity.

LUMINEX™ multifunctional liquid-phase chip analyses of GM-CSF, TNF-α, IL-1 and IL-6: The 96-well plate was enclosed with enclosing liquid for 30 minutes. Fluorescent microspheres were added into the 96-well plate after dilution. The standard and the samples to be measured were added, and incubated overnight at 4° C. The next day, the supernatant was discarded and 50 μL GM-CSF, TNF-α, IL-1β and IL-6 antibodies were added to each well. After being rinsed for 4 times, the 96-well plate was placed in a shaking table, which was in ambient temperature, to keep the plate away from light for one hour. Again, the plate was rinsed for 4 times, and PE labeled with streptavidin was added. The plate was kept in the dark away from light for 15 minutes at room temperature, and then it was rinsed for 4 times. The microspheres were suspended in the rinsing buffer, and the 96-well plate placed immediately on the LUMINEX™ (Luminex Corporation, Austin, Tx.) machine for analysis.

The compound Morusin A has a very strong anti-inflammatory ability. The specific results are shown in Table 3.

TABLE 3
the anti-inflammatory effects of Morusin A (the larger the value,
the stronger the anti-inflammatory ability)

| Extract/compound | GM-CSF | IL-1β | IL-6 | TNF-α |
|---|---|---|---|---|
| Compound Morusin A | 11% | 23% | 53% | 93% |
| of formula (I) | | | | |
| Triclosan (Positive | 5% | 7% | 31% | 81% |
| control) | | | | |

The above experimental results indicate that Morusin A has excellent inhibitory effect on oral pathogenic microorganisms (Actinomyces viscosus, Actinobacillus actinomycetemcomitans, Streptococcus mutans, Porphyromonas gingivalis), has significant anti-inflammatory activity to oral KB cells, and also has significant inhibitory effect on inflammatory factors; and therefore should be very useful in the formulation of oral care products and pharmaceuticals for the treatment of bacterial and inflammatory diseases.

### EXAMPLE 5

### Morusin A Uses

Morusin A may be used directly, or may be employed in oral pharmaceuticals or oral care products such as toothpaste, mouthwash, paste, tablets, thin film, chewing gum and the like as the effective ingredient. It may be in an effective amount together with other ingredients such as humectant, abrasive, surfactant, pharmaceutically acceptable carrier and/or additive, colorants, stabilizers, flavorants, sweeteners, and it may include other pharmaceutical agents such as antibacterial.

The term “effective amount” indicated herein refers to an amount sufficient to produce a positive effect. Such an effective amount will vary in particular embodiments; for example, a gum may contain less active ingredient that a mouthwash since it may be used several times a day. In addition, there may be multiple effective dosages or an effective dosage range. In some examples, Morusin A may be mixed with other ingredients in an amount of 0.0005-99% (all % are mass percentage unless otherwise indicated). In some other embodiments, the content of 5 Morusin A is 0.001-10%. In some preferred examples, the content of Morusin A is 0.025-5%. In some other examples, the content of Morusin A is 0.05-0.5%. Yet in other examples, the content of Morusin A is 0.5-90%.

Preferably, when used in a tablet formulation, the content of Morusin A is 4%; in nasal sprays, the content of Morusin A is 4%; in dropping pills, the content of Morusin A is 10%; in toothpastes, the content of Morusin A is 0.5%; in mouthwash, the content of Morusin A is 0.5%, and in oral pastes, the content of Morusin A is 0.01%. Other effective amounts may also be applicable in the present invention.

The composition of formulation according to the present invention may also contain cationic surfactants and/or non-ionic surfactants. Cationic surfactants include, but not limited to, cetyltrimethylammonium chloride, di(isobutyl-phenoxy-ethoxy-ethyl)-di(xylene-benzyl) ammonium bromide. Non-ionic surfactants include Poloxamer, polyethoxy ether, and ethoxy fatty acid and the like. Poloxamer is a block copolymer of polyoxyethylene and polyoxypropylene, having industrial applications, such as that produced by BASF under the trade name “Pluronic”. Polyethoxy ether includes polyoxyethylene sorbitol fatty acid ester (a typical monoester of polyethoxy alcohol), which is produced by ICL company with the trade name Tween. Other non-ionic surfactants include polyoxyethylene alkylphenol, polyoxyethylene alcohols, fatty acids, polyoxyethylene esters, polyoxyethylene alkyl amine glyceride, polyglyceride, tetritol ester, pentitol ester, hexitol ester, dehydrated tritol ester and polyoxyalkylamine polyalkyl ester. Typically, the mass content of surfactant in the formulation is 0.001%-3.0%. Divalent metal ions, such as zinc, copper, selenium, calcium or magnesium, may be included in the formulation. They will be in the form of soluble inorganic salts such as zinc chloride or the form of organic/inorganic compounds. The amount of divalent metal ions added into the mixture will be 0.001%-3.0%. The formulation may contain oligosaccharides, which will be in the water-soluble state, and may convert into soluble salt, if added, in an amount of at least 0.01%.

The present invention may include various commonly used oral pharmaceuticals or oral hygiene products, such as mouthwash, oral spray, toothpaste, gels, oral mucosa plasters, oral pellicle, instant tablet and film, chewing gum and the like products for mammals, especially human and animals, to inhibit plaque and prevent dental caries and periodontal disease.

Other additives may be appropriately used for the embodiment—mouthwash may contain acidic substances; mouthwash may also contain desensitizing substance such as potassium nitrate; toothpaste may contain abrasive such as sodium carbonate, calcium phosphate, alumina and silica; solubilizer such as PEG, glycerol, ethanol; other taste masking agents such as xylitol; thickener such as carrageenan; and surfactant such as SLS, etc. may all be used.

Waxes such as beeswax may be used in toothpaste or oral mucosa plaster; thickener and pellicle-forming agents such as carrageenan, CMC, HPMC, xanthan gum and the like may also be added.

Final products may include essences such as mint, spearmint, eucalyptus leaf oil, menthol, carvone, wintergreen, cloves, cinnamon, lemon, grapefruit, orange and some essences decomposed and digested from yeast and protein.

In tablets and pellicles, inert excipients may be used for the formation of the products. Adjuvants such as colorant, taste masking agent and the like are mainly included to improve the taste and appearance.

The composition of the formulation related with the application of the present invention in tablets may contain filler, adhesive, lubricant, disintegrant. Fillers include, but not limited to, starch, lactose, microcrystalline cellulose and the like. Adhesives include, but not limited to, starch slurry, cellulose derivatives, polyvidone, gelatin and the like. Lubricants include, but not limited to, magnesium stearate, micropowder silica gel, talcum powder, polyethylene glycols and the like. Disintegrants include, but not limited to, sodium carboxymethyl starch, low substituted hydroxylpropyl cellulose, cross-linked polyvidone, cross-linked sodium carboxymethyl cellulose and the like. All these ingredients are pharmaceutically acceptable adjuvants recorded in the Chinese Pharmacopoeia and the Pharmacopoeia of many countries such as United Kingdom and United States.

Matrix such as polyethylene glycol and the like may be used in the pastes of the present invention. Polyethylene glycol in solid may be combined with liquid forms to adjust the consistency. Ingredients such as magnesium stearate may also be added to adjust the consistency.

### EXAMPLE 6

### Specific Formulations

Specific formulations are presented below:

### Morusin A Toothpaste Formulation 1:

| Components | Content (mass percentage, %) |
|---|---|
| Morusin A | 0.0005-20 |
| Abrasive | 10-30 |
| Humectant | 50-69 |
| Water, Flavor, Flavoring | proper amount |

### Morusin A Toothpaste Formulation 2:

| Components | Content (mass percentage, %) |
|---|---|
| Morusin A | 5 |
| Silica | 30 |
| Glycerol | 5 |
| Sorbitol | 50 |
| Water, Flavor, Flavoring | proper amount |

### Morusin A Toothpaste Formulation 3:

| Components | Content (mass percentage, %) |
|---|---|
| Morusin A | 0.001 |
| Silica | 10 |
| Sorbitol | 69 |
| Water, Flavor, Flavoring | proper amount |

### Morusin A Toothpaste Formulation 4:

| Components | Content (mass percentage, %) |
|---|---|
| Morusin A | 10 |
| Abrasive | 25 |
| Humectant | 65 |

### Morusin A Toothpaste Formulation 5:

| Components | Content (mass percentage, %) |
|---|---|
| Morusin A | 0.0005 |
| Silica | 10 |
| Sorbitol | 70 |
| Water, Flavor, Flavoring | proper amount |

### Morusin A Toothpaste Formulation 6:

| Components | Content (mass percentage, %) |
|---|---|
| Morusin A | 20 |
| Abrasive | 25 |
| Humectant | 55 |

### Morusin A Toothpaste Formulation 7:

| Components | Content (mass percentage, %) |
|---|---|
| Morusin A | 0.025 |
| Silica | 20 |
| Glycerol | 5 |
| Sorbitol | 60 |
| Sodium fluoride | 0.221 |
| Sodium saccharin | 0.3 |
| Water | 7.954 |
| Polyethylene glycol | 3 |
| Sodium dodecyl sulfate | 2 |
| Essence | 1.5 |

### Morusin A Toothpaste Formulation 8:

| Components | Content (mass percentage, %) |
|---|---|
| Morusin A | 0.3 |
| Silica | 20 |
| Glycerol | 5 |
| Sorbitol | 60 |
| Sodium fluoride | 0.221 |
| Sodium saccharin | 0.3 |
| Water | 7.679 |
| Polyethylene glycol | 3 |
| Sodium dodecyl sulfate | 2 |
| Essence | 1.5 |

### Morusin A Toothpaste Formulation 9:

| Components | Content (mass percentage, %) |
|---|---|
| Morusin A | 0.5 |
| Silica | 20 |
| Glycerol | 5 |
| Sorbitol | 60 |
| Sodium fluoride | 0.221 |
| Sodium saccharin | 0.3 |
| Water | 7.479 |
| Polyethylene glycol | 3 |
| Sodium dodecyl sulfate | 2 |
| Essence | 1.5 |

### Morusin A Mouthwash Formulation 1:

| Components | Content (mass percentage, %) |
|---|---|
| Morusin A | 0.0005-20 |
| Water | 70-94 |
| other additives | proper amount |

### Morusin A Mouthwash Formulation 2:

| Components | Content (mass percentage, %) |
|---|---|
| Morusin A | 5 |
| Water | 80 |
| Pluronics | 3.00 |
| other additives | proper amount |

### Morusin A Mouthwash Formulation 3:

| Components | Content (mass percentage, %) |
|---|---|
| Morusin A | 0.001 |
| Water | 94 |
| Alcohol | 5.00 |
| other additives | proper amount |

### Morusin A Mouthwash Formulation 4:

| Components | Content (mass percentage, %) |
|---|---|
| Morusin A | 0.5 |
| Water | 90 |
| Pluronics | 3.00 |
| Alcohol | 3.00 |
| other additives | proper amount |

### Morusin A Mouthwash Formulation 5:

| Components | Content (mass percentage, %) |
|---|---|
| Morusin A | 0.025 |
| Water | 85 |
| Pluronics | 5.00 |
| Alcohol | 3.00 |
| other additives | proper amount |

### Morusin A Mouthwash Formulation 6:

| Components | Content (mass percentage, %) |
|---|---|
| Morusin A | 0.05 |
| Water | 91.7 |
| Pluronics | 3.00 |
| Alcohol | 5.00 |
| Essence | 0.25 |

### Morusin A Mouthwash Formulation 7:

| Components | Content (mass percentage, %) |
|---|---|
| Morusin A | 0.0005 |
| Water | 94 |
| Alcohol | 5.00 |
| other additives | proper amount |

### Morusin A Mouthwash Formulation 8:

| Components | Content (mass percentage, %) |
|---|---|
| Morusin A | 20 |
| Water | 70 |
| Pluronics | 3.00 |
| Alcohol | 3.00 |
| other additives | proper amount |

### Morusin A Paste Formulation 1:

| Components | Content (mass percentage, %) |
|---|---|
| Morusin A | 10 |
| Polyethylene glycol | 90 |

### Morusin A Paste Formulation 2:

| Components | Content (mass percentage, %) |
|---|---|
| Morusin A | 0.001 |
| Polyethylene glycol | 80 |
| other additives | proper amount |

### Morusin A Paste Formulation 3:

| Components | Content (mass percentage, %) |
|---|---|
| Morusin A | 0.01 |
| Polyethylene glycol | 90 |
| other additives | proper amount |

### Morusin A Paste Formulation 4:

| Components | Content (mass percentage, %) |
|---|---|
| Morusin A | 5 |
| Polyethylene glycol | 95 |

### Morusin A Paste Formulation 5:

| Components | Content (mass percentage, %) |
|---|---|
| Morusin A | 0.025 |
| Polyethylene glycol | 99.975 |

### Morusin A Paste Formulation 6:

| Components | Content (mass percentage, %) |
|---|---|
| Morusin A | 20 |
| Polyethylene glycol | 80 |

### Morusin A Paste Formulation 7:

| Components | Content (mass percentage, %) |
|---|---|
| Morusin A | 0.0005 |
| Polyethylene glycol | 99 |
| other additives | proper amount |

Morusin A Tablets—Morusin A 10 mg, lactose 180 mg, starch 55 mg, magnesium stearate 5 mg. Active ingredient, lactose and starch, are mixed and wetted uniformly with water. The wetted mixture is subjected to sieve and dry, subjected to sieve again. Then magnesium stearate is added in. The mixture is then compressed into tablets, weighing 250 mg each, with a final content of the active ingredient content of 10 mg per tablet.

Morusin A Nasal Spray:

| Components | Content | |
|---|---|---|
| Morusin A | 80 | mg |
| sodium chloride | 8 | mg |
| EDTA | 1 | mg |
| Sodium phosphate buffer (pH 6.5) | 10 | mg |
| Polyethoxy ether | 10 | mg |
| redistilled water | to 2 | ml |

Preparing method: One ingredient at a time is added into the proper volume of redistilled water while being stirred until it is completely dissolved, then another ingredient is added. After adding water to 2 ml, the solution is filtered on a sterile filter into bottles and separated according to suitable dosage.

Morusin A Dropping Pills:

| Components | Content |
|---|---|
| Morusin A | 1 g |
| polyethylene glycol 6000 | 9 g |

Preparing method: preparation of molten liquid of Morusin A and polyethylene glycol 6000: Morusin A is weighed according to the prescribed amount above, to which a proper amount of dehydrated ethanol is added. After being dissolved by tepefaction, the resulting substances are added into the prescribed amount of polyethylene glycol molten liquid (incubated in 60° C. water bath). The mixture is stirred to mix uniformly until the ethanol is completely evaporated. The resultant is allowed to stay in 60° C. water bath to incubate for 30 minutes. After the bubbles are completely removed from the uniformly mixed molten liquid above, the liquid is transferred into a storing tank. Under a condition where the temperature is maintained at 80-85° C., the molten liquid is added drop-by-drop into a condensing liquid under a controlled dropping speed. After the condensation is completed, the condensing liquid is decanted and the drop pills are collected. The condensing liquid on the pills is trickled and removed with filter paper. Then, the pills are placed in a silica gel desiccators or dried naturally.

The above detailed description of the present invention sets forth a lot of specified details. The object thereof is to give clear and integrated explanation of the invention to facilitate the public to read and understand. However, what will be clear to one skilled in the art is that in some cases, the present invention may also be practiced without these specified details, or by making some non-essential modification or substitution to these specified details. These variations should be considered to fall within the spirit and scope of the present invention. The scope of the present invention is determined by the plain language of the appended claims, otherwise limited by the specific examples provided in the detailed description.

1. A purified compound of formula (I):

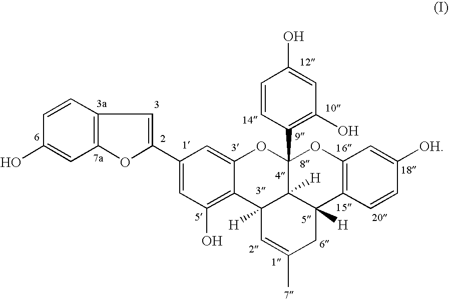

2. The purified compound of claim 1, wherein the compound is extracted from a plant of the genus Morus.

3. The purified compound of claim 1, wherein the compound is extracted from Cortex Mori.

4. The purified compound of claim 1, wherein the purity of the compound is at least 50%.

5. The purified compound of claim 1, wherein the purity of the compound is at least 75%.

6. The purified compound of claim 1, wherein the purity of the compound is at least 95%.

7. The purified compound of claim 1, wherein the purity of the compound is at least 98%.

8. The purified compound of claim 1, wherein the purity of the compound is at least 99%.

9. A composition comprising the purified compound of formula (I) in an amount effective as an oral care product, pharmaceutical composition, antibacterial composition or the like, and an acceptable carrier:

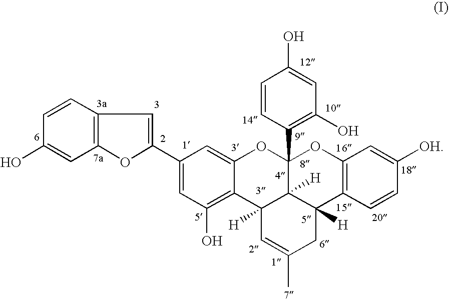

10. The oral care product of claim 9, wherein the final content of the purified compound of formula (I) is at least 0.0005%.

11. The oral care product of claim 9, wherein the final content of the purified compound of formula (I) is 0.001-10%.

12. The oral care product of claim 9, wherein the final content of the purified compound of formula (I) is 0.025-5%.

13. The oral care product of claim 9, wherein the final content of the purified compound of formula (I) is 0.05-0.5%.

14. The pharmaceutical composition of claim 9, comprising the purified compound of formula (I) in an amount effective for treating or preventing bacterial or inflammatory disease, and a pharmaceutically acceptable carrier.

15. The pharmaceutical composition of claim 14, wherein the final content of the purified compound of formula (I) is at least 0.05%.

16. The pharmaceutical composition of claim 14, wherein the final content of the purified compound of formula (I) is 0.1-50%.

17. The antibacterial composition of claim 9, comprising the purified compound of formula (I) in an amount effective for killing bacteria or preventing the growth of bacteria, together with a carrier.

18. The antibacterial composition of claim 17, wherein the final content of the purified compound of formula (I) is at least 0.0005%.

19. The antibacterial composition of claim 17, wherein the final content of the purified compound of formula (I) is 0.01-10%.

20. A use of the purified compound of formula (I) in manufacturing oral care products

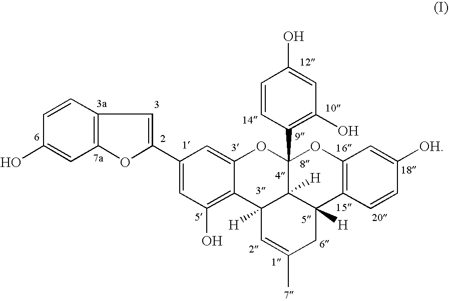

21. The use of claim 20, wherein the final content of the purified compound of formula (I) is at least 0.0005%.

22. The use of claim 20, wherein the final content of the purified compound of formula (I) is 0.001-10%.

23. The use of claim 20, wherein the final content of the purified compound of formula (I) is 0.025-5%.

24. The use of claim 20, wherein the final content of the purified compound of formula (I) is 0.05-0.5%.

25. A method of purifying the purified compound of formula (I), comprising extracting root and/or bark from Cortex Mori with 90% ethanol, then with ethyl acetate, and then dissolving the extract in acetone and subjecting the acetone extract to silica gel chromatography and elution with petroleum ether: acetone in 8:2 (v/v)

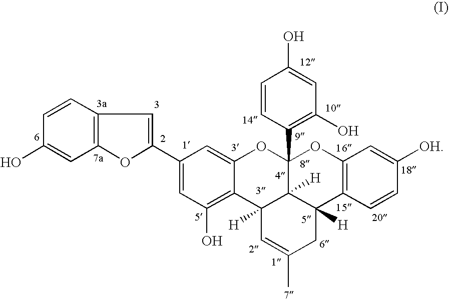

26. The method of claim 17, wherein said purified compound of formula (I) is at least 75% pure.

27. The method of claim 17, wherein said purified compound of formula (I) is at least 90% pure.

28. The method of claim 17, wherein said purified compound of formula (I) is at least 98% pure.
